(19) United States
(12) Patent Application Publication
(10) Pub. No.: US 2006/0177424 A1
(43) Pub. Date: Aug. 10, 2006

(54) Treatment of disease states and adverse physiological conditions utilizing anti-fungal compositions

(76) Inventors: Mark L. Cobb, (US); Alyson J. Cobb, (US)

Correspondence Address: INTELLECTUAL PROPERTY / TECHNOLOGY LAW, PO BOX 14329, RESEARCH TRIANGLE PARK, NC, 27709, US

(21) Appl. No.: 10/928,951

(22) Filed: Aug. 27, 2004

Related U.S. Application Data
(60) Provisional application No. 60/499,255, filed on Aug. 29, 2003.

(51) Int. Cl. A61K 35/74

(52) U.S. Cl. 424/93.4; 424/93.45

(57) ABSTRACT

A method for treatment or prophylaxis of a disease state or other physiological condition, e.g., autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, asthma, attention deficit disorder, attention deficit/hyperactivity disorder, rosacea, multiple sclerosis, hyperglycemia, or Ménière's disease, by administration of an anti-fungal composition that includes at least one of the bacilli (1) Bacillus subtilis, (2) Lactobacillus sporogenes, and (3) Streptococcus faecalis.

### CROSS-REFERENCE TO RELATED APPLICATIONS

This application claims the benefit under 35 U.S.C. §119(e) of U.S. Provisional Patent Application No. 60/499,255 filed on Aug. 29, 2003 in the names of Mark L. Cobb and Alyson J. Cobb for “TREATMENT OF DISEASE STATES AND ADVERSE PHYSIOLOGICAL CONDITIONS UTILIZING ANTI-FUNGAL COMPOSITIONS,” the contents of which are incorporated by reference herein for all purposes.

### BACKGROUND OF THE INVENTION

The present invention in a broad aspect relates to a method for treatment of disease states and/or other adverse physiological conditions, and more specifically to a method for treating a patient suffering from such a disease state or other adverse physiological condition, comprising the administration of an effective dose of an anti-fungal bacteria composition to a patient in need of treatment thereof.

### Description of the Related Art

Candidiasis is a fungal infection of mucosal membranes and other tissues. The infection is caused by the yeast-like organism Canidida. Numerous species of Candida exist, including C. albicans. The recent increase in candidiasis is most likely caused by the rising incidence of AIDS, more intensive regimens of cancer therapy, complications of abdominal or cardio-thoracic surgery, organ transplantations, burns and trauma. In addition, immunocompromised individuals and women of childbearing age, especially pregnant women or women with one or more childbirths, are known to be more susceptible to microbial pathogenesis. Alteration of the fungi microenvironment, including changes in pH, temperature, osmotic pressure, and hormonal concentrations, is currently considered to be accountable for the initiation of C. albicans infection symptoms.

Women can contract vaginal candidiasis by engaging in sexual activity with men who may not be aware they are carrying the infection because the symptoms are so mild in men that they are either overlooked or are completely unfelt.

It has recently come to light that candidiasis strikes immunocompetent individuals as well. For example, the widespread use of anti-microbial agents, such as broad spectrum antibiotics, has resulted in a number of serious clinical consequences. For example, antibiotics can kill beneficial, non-pathogenic microorganisms (i.e., flora) within the gastrointestinal tract, but are powerless against the yeasts in the GI tract. As a result, the gastrointestinal yeasts normally kept in check by the flora begin to grow at an excessive rate. In addition to antibiotics, the unrestrained usage of steroids, birth control pills, antacid and anti-ulcer medications, as well as diets high in sugar have been blamed for the rapid rise in candidiasis in the general population.

Common candidiasis symptoms include, but are not limited to, vaginal yeast infections, psoriasis and other dermatosis, fatigue or lethargy, depression, headaches, muscle aches, pain and/or swelling in the joints, constipation and/or diarrhea, bloating, sexual dysfunction, chronic hives, irritability, PMS, arthritis, muscle pain, memory loss, vaginitis, impotence, hypoglycemia, menstrual problems, urinary disorders, anxiety, insomnia, sensitivity to foods, thrush, and halitosis.

It is theorized that approximately 85% of Americans are or have been infected by some strain of yeast or fungus. Fungal infections can be systemic, subcutaneous, cutaneous or superficial (involving the outermost skin or hair).

Fungal infections are among the most difficult to effectively treat and regimens must be continued for months before results can be seen. Currently, there are several different types of drugs on the market that can provide effective anti-fungal therapy, as well as anti-fungal diets, such as the four step Candida diet. The Candida diet includes: the immediate elimination of antibiotics, birth control pills and other hormone-altering substances; a low-sugar, low-carbohydrate, high protein diet; the medical use of anti-fungal agents such as nystatin; and the strengthening of the immune system through supplementation. Though often effective, anti-fungal drugs and the Candida diet are cost prohibitive to many and more often than not, the symptoms return upon termination of the regimen.

To date, the cause of fibromyalgia syndrome, which causes people to feel chronic pain all over their bodies and suffer from symptoms such as sleep disturbances, fatigue and headaches, is unknown. It has been reported that an estimated four to six million Americans suffer from fibromyalgia, however presently there is no drug designed specifically to treat it.

Like fibromyalgia, the cause of autism continues to evade researchers. There are numerous theories put forth to explain the onset of autism in children including, but not limited to: an excess of opioid-like species whose effects on the brain produce the symptoms of autism; vaccinations, e.g., MMR; genetics; toxic environmental substances; and difficulties during pregnancy.

It would be beneficial to provide an affordable treatment that would prevent or inhibit the unchecked growth of fungus, including Candidiasis, which is also effective to treat, prevent, ameliorate or regulate other disease states and adverse physiological conditions including, but not limited to, autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, asthma, attention deficit disorder, attention deficit/hyperactivity disorder, rosacea, multiple sclerosis, hyperglycemia, and Ménière's disease.

### SUMMARY OF THE INVENTION

In one aspect, the present invention relates to a method of treating disease states and/or other adverse physiological conditions comprising administering, to a subject in need thereof, an effective amount of a composition comprising at least one bacterial species effective in ameliorating or regulating the disease state or physiological condition.

In another aspect, the present invention relates to a method of treating disease states and other adverse physiological conditions comprising administering, to a subject in need thereof, at least one lactic acid producing bacteria and at least one soil based bacteria in a sufficient ratio to effectively ameliorate or regulate the disease state or physiological condition.

In yet another aspect, the present invention relates to a method of treatment or prophylaxis of a disease state or a physiological condition selected from the group consisting of autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, attention deficit disorder, attention deficit/hyperactivity disorder, rosacea, multiple sclerosis, hyperglycemia, and Ménière's disease, said method comprising administering to a subject in need thereof, an effective amount of a composition comprising at least one of (1) Bacillus subtilis, (2) Lactobacillus sporogenes, and (3) Streptococcus faecalis, with the proviso that when the disease state is autism, colitis or Crohn's Disease, the composition comprises Bacillus subtilis, Lactobacillus sporogenes, and Streptococcus faecalis.

In a further aspect, the present invention relates to a method of treatment or prophylaxis of a disease state or a physiological condition selected from the group consisting of delayed development, acid reflux disease, impaired hearing, chronic ear infections, seasonal allergies, Fibromyalgia syndrome, irritable bowel syndrome, attention deficit disorder, attention deficit/hyperactivity disorder, rosacea, multiple sclerosis, hyperglycemia, and Ménière's disease, said method comprising administering to a subject in need thereof, an effective amount of a composition comprising at least one of (1) Bacillus subtilis, (2) Lactobacillus sporogenes, and (3) Streptococcus faecalis.

A still further aspect of the invention relates to a method of treatment or prophylaxis of a disease state or a physiological condition selected from the group consisting of autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, attention deficit disorder, attention deficit/hyperactivity disorder, rosacea, multiple sclerosis, hyperglycemia, and Ménière's disease, said method comprising administering to a subject in need thereof, an effective amount of a composition comprising at least one of (1) Bacillus subtilis, (2) Lactobacillus sporogenes, and (3) Streptococcus faecalis, wherein the method of administration is selected from the group consisting of oral, subcutaneous, intramuscular, intradermal, transdermal, intraocular, intraperitoneal, mucosal, vaginal, rectal, and intravenous, and when the disease state is autism, colitis or Crohn's Disease, the composition comprises Bacillus subtilis, Lactobacillus sporogenes, and Streptococcus faecalis.

Yet another aspect of the present invention relates to a method of treatment or prophylaxis of a disease state or a physiological condition selected from the group consisting of autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, asthma, attention deficit disorder, attention deficit/hyperactivity disorder, rosacea, multiple sclerosis, hyperglycemia, and Ménière's disease, said method comprising administering to a subject in need thereof, an effective amount of a composition comprising at least one of (1) Bacillus subtilis, (2) Lactobacillus sporogenes, and (3) Streptococcus faecalis, with the proviso that when the disease state or physiological condition comprises asthma, the composition is administered by an administration modality not including mucoadministration to the airways of the subject.

Another aspect of the invention relates to a method of treatment or prophylaxis of autism, said method comprising administering to a subject in need thereof, an effective amount of a composition comprising (1) Bacillus subtilis, (2) Lactobacillus sporogenes, and (3) Streptococcus faecalis.

Other aspects, features and embodiments of the invention will be more fully apparent from the ensuing disclosure and appended claims.

### DETAILED DESCRIPTION OF THE INVENTION

The present invention relates to a method for treatment of disease states and other adverse physiological conditions, and more specifically to methods for treating a patient suffering from disease states or other adverse physiological conditions comprising the administration of an effective dose of an anti-fungal bacteria composition to a patient in need of treatment thereof.

Applicants have unexpectedly discovered that the anti-fungal compositions of the present invention are able to effectively ameliorate and/or regulate several maladies including, but not limited to, autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, asthma, attention deficit disorder, attention deficit/hyperactivity disorder, rosacea, multiple sclerosis, hyperglycemia, and Ménière's disease.

For example, applicants have surprisingly discovered that the anti-fungal composition of the present invention effectively ameliorates or regulates the symptoms of fibromyalgia. The term “effectively ameliorates or regulates” refers to substantial improvement in the symptoms associated with the subject disease state or physiological condition, e.g., at least 50% improvement in such symptoms. Additionally, applicants have discovered that when autistic patients were administered the anti-fungal composition of the present invention, their manifested symptoms (i.e., minimal eye contact, verbal skills, etc.) were lessened in severity. In many cases, the symptoms of autism were no longer present and the diagnosis was removed.

The gastrointestinal microflora has been shown to play a number of vital roles in maintaining gastrointestinal tract function and overall physiological health. For example, the growth and metabolism of the many individual bacterial species inhabiting the gastrointestinal tract depend primarily upon the substrates available to them, most of which are derived from the diet. See e.g., Gibson G. R. et al., 1995. Gastroenterology 106: 975-982; Christi, S. U. et al., 1992. Gut 33: 1234-1238. These findings have led to attempts to modify the structure and metabolic activities of the community through diet, primarily with probiotics, which are live microbial food supplements. Probiotic microorganisms are those which confer a benefit when grown in a particular environment, often by inhibiting the growth of other biological organisms in the same environment. The best known probiotics are the lactic acid-producing bacteria (i.e., Lactobacilli) and Bifidobacteria, which are widely utilized in yogurts and other dairy products. These probiotic organisms are non-pathogenic and non-toxigenic, retain viability during storage, and survive passage through the stomach and small intestine. Since probiotics do not permanently colonize the host, they need to be ingested regularly for any health promoting properties to persist. Commercial probiotic preparations are generally comprised of mixtures of Lactobacilli and Bifidobacteria, although yeast such as Saccharomyces has also been utilized.

The anti-fungal bacteria composition of the present invention includes lactic-acid producing bacteria and soil bacteria. Specifically, the bacilli Bacillus subtilis, Streptococcus faecalis, and Lactobacillus sporogenes, or a combination thereof, are contemplated herein. Specifically, in the broad practice of the invention, the anti-fungal bacteria composition of the present invention may comprise, consist of, or consist essentially of one or more of Bacillus subtilis, Streptococcus faecalis, and Lactobacillus sporogenes. An illustrative embodiment of the anti-fungal composition of the invention includes the anti-fungal bacteria composition sold under the trademark THREELAC, commercially available from Global Health Trax (Vista, Calif., USA).

The Bacillus species, particularly those species having the ability to form spores (e.g., Bacillus coagulans), are a preferred embodiment of the present invention. The ability to sporulate makes these bacterial species relatively resistant to heat and other conditions, provides for a long shelf-life in product formulations, and is ideal for survival and colonization of tissues under conditions of pH, salinity, and the like within the gastrointestinal tract. Additional useful properties of many Bacillus species include being non-pathogenic, aerobic, facultative and heterotrophic, thus rendering these bacterial species safe and able to readily colonize the gastrointestinal tract.

It should be noted that Lactobacillus sporogenes has recently been re-characterized as Bacillus coagulans. The initial classification as Lactobacillus sporogenes (see, Nakamura et al., Int. J. Syst. Bacteriol., 38: 63-73, 1988) has been argued as incorrect due to the fact that the Lactobacillus sporogenes strain produces spores and through metabolic processes excretes L(+)-lactic acid, which under current classification rules requires that the bacterium be classified as a bacillus species. Furthermore, classic Lactobacillus species are normally unsuitable for colonization of the gut due to their instability in the harsh (i.e., acidic) pH environment of the bile, particularly human bile, but Lactobacillus sporogenes is able to survive and colonize the gastrointestinal tract. Though re-characterized, the common name Lactobacillus sporogenes will be used hereinafter instead of Bacillus coagulans.

Lactobacillus sporogenes is a non-pathogenic, Gram positive, spore-forming bacteria that produces L(+) lactic acid (dextrorotatory) under homo-fermentation conditions. It has been isolated from natural sources, such as heat-treated soil samples inoculated into nutrient medium (see e.g., Bergey's Manual of Systemic Bacteriology, Vol. 2, Sneath, P. H. A. et al., eds., Williams & Wilkins, Baltimore, Md., 1986). Lactobacillus sporogenes has also been utilized to produce lactic acid (U.S. Pat. No. 5,079,164). Though not naturally found in the gut, Lactobacillus sporogenes strains have been used as general nutritional supplements and agents to control constipation and diarrhea in humans and other animals.

Lactobacillus sporogenes strains and their growth requirements have been described previously (see e.g., Baker, D. et al, Can. J. Microbiol., 6: 557-563, 1960; Nakamura, H. et al, Int. J Syst. Bacteriol. 38: 63-73, 1988). In addition, various strains of Lactobacillus sporogenes can be isolated from natural sources (e.g., heat-treated soil samples) using well-known procedures (see e.g., Bergey's Manual of Systemic Bacteriology, Vol. 2, p. 1117, Sneath, P. H. A. et al., eds., Williams & Wilkins, Baltimore, Md., 1986).

Bacillus subtilis is a Gram-positive, endospore-forming soil bacterium comprising aerobic and a few facultatively anaerobic rod-shaped bacteria. Bacillus subtilis was historically used to treat dysentery. It has been reported that ingested Bacillus subtilis are able to activate the human immune defense, including the IgM, IgG and IgA antibodies. Bacillus subtilis strains and their growth requirements are well known in the art.

Streptococcus faecalis is a facultative anaerobic, Gram positive, cocci that produces L(+) lactic acid. The Streptococcus faecalis (more correctly referred to as Enterococcus faecalis) strain is a natural inhabitant of the mammalian G.I. tract and causes many of the same problems as other members of the intestinal flora, including opportunistic urinary tract infections and wound infections.

The compositions of the invention comprising the bacilli (A) Bacillus subtilis, (B) Streptococcus faecalis, and (C) Lactobacillus sporogenes, or any combination thereof, are suitably administered to a subject in need of treatment in an amount sufficient to elicit an anti-fungal response or alternatively, to enhance an ongoing anti-fungal response. The compositions of the invention can also be administered to a subject in need of treatment in an amount sufficient to effectively ameliorate and/or regulate symptoms associated with other non-fungally related disease states or physiological conditions.

The invention contemplates compositions comprising the bacilli Bacillus subtilis (“A”), Streptococcus faecalis (“B”), and Lactobacillus sporogenes (“C”), in which each of such bacilli A, B and C may vary in relative amount with respect to one another, in various embodiments of the invention. For example, each of A, B and C may be present in an equal or equivalent amount, in relation to one another. In other embodiments, A, B and C may be present in unequal amounts in relation to one another, with each of A, B and C being present in an amount in a range of from about 5% to about 90% by weight, based on the total weight of A, B and C, and with all amounts of A, B and C totalling 100%. Thus, the invention contemplates a wide variety of specific formulation embodiments, icluding by way of example the following illustrative compositions (showing percentages by weight for each of the A, B and C ingredients, based on the total weight of A+B+C in the composition):

| Weight | | | | |
|---|---|---|---|---|
| percentages for | | | | Total wt |
| each of A, B, C | | | | % of A, |
| in Composition No: | A wt % | B wt % | C wt % | B and C |
| 1 | 30 | 30 | 40 | 100 |
| 2 | 25 | 40 | 35 | 100 |
| 3 | 25 | 50 | 25 | 100 |
| 4 | 25 | 70 | 5 | 100 |
| 5 | 20 | 40 | 40 | 100 |
| 6 | 20 | 50 | 30 | 100 |
| 7 | 20 | 60 | 20 | 100 |
| 8 | 10 | 50 | 40 | 100 |
| 9 | 10 | 60 | 30 | 100 |
| 10 | 10 | 80 | 10 | 100 |
| 11 | 5 | 80 | 15 | 100 |
| 12 | 5 | 90 | 5 | 100 |

The specific relative amounts of the active ingredients in the compositions of the invention will depend on a variety of factors known to those of skill in the art of therapeutic formulation. For example, factors to be considered include the route of administration and the nature of the subject to be treated. The effect of such factors, and other factors known in the art such as synergistic effects, is readily determined by one of skill in the art according to standard clinical techniques. Effective doses of the active components of the compositions of the present invention may also be extrapolated from dose-response curves derived from animal model test systems. Therapeutic results accumulated to date suggest that the gender and age of the subject are irrelevant to the overall efficacy of the composition, with the exception of patients less than six years of age, who are generally more responsive to the composition than any other age group.

The composition of the invention may be administered in any suitable dose amount that is effective to prevent, ameliorate, regulate, cure or otherwise treat the disease state or physiological condition in a subject in need of such therapeutic intervention.

In various specific embodiments, an effective dose of the anti-fungal composition of the present invention is about 1.0 g to about 15.0 g for an adult patient, more preferably between about 2.0 g and about 10.0 g. Effective doses are to be administered to a patient in need at least once a week, preferably once a day. Pediatric dosages may be in the range of 15% to 90% of adult dosages.

For example, some patients may administer a constant dosage of the anti-fungal composition over time, for example about 2 g to about 4 g per day, while some patients may choose to increase the dosage up to about 6 g to about 10 g per day, depending on the severity of the disease state or physiological condition. Once the disease state or physiological condition has been effectively ameliorated or regulated, the patient may decrease the dosage to about 2 g to about 4 g per day for maintenance purposes.

In general, the effective dosage of compositions of the invention for therapeutic use may be widely varied in the broad practice of the invention, depending on the specific application, condition, or disease state involved, as readily determinable within the skill of the art. By way of illustration, in some embodiments of the invention, the effective dosage of the composition may be in a range of from about 10 milligrams (mg) to 200 milligrams (mg) per kilogram body weight of the recipient per day. The desired dose may be presented in multiple (e.g., two, three, four, five, six, or more) sub-doses administered at appropriate intervals throughout the day. These sub-doses may be administered in unit dosage forms containing an appropriate amount of active ingredients per unit dosage form.

The anti-fungal compositions of the invention may additionally and optionally comprise any suitable adjuvants, excipients, additives, carriers, additional therapeutic agents, bioavailability enhancers, side-effect suppressing components, or other ingredients that do not preclude the efficacy of the composition for ameliorating or regulating the disease states or physiological conditions. Preferably, the bacilli (Bacillus subtilis, Streptococcus faecalis, and Lactobacillus sporogenes) comprise from about 50% to about 90% by weight of the composition, based on the total weight of the anti-fungal composition of the invention. Most preferably, the bacilli comprise from about 60% to about 80% by weight of the composition.

The compositions of the present invention suitable for oral administration may be presented as discrete units such as gelatin capsules or cachets, each containing a predetermined amount of the anti-fungal bacteria composition as a powder or granules.

Other routes of administration are contemplated including, but not limited to, subcutaneous, intramuscular, intradermal, transdermal, intraocular, intraperitoneal, mucosal, vaginal, rectal, and intravenous. For example, a dose of the anti-fungal composition of the present invention may be inserted in a gelatin capsule and inserted vaginally to treat vaginal yeast infections.

When the disease state is asthma, the route of administration can be any of those listed hereinabove, with the exception of mucoadministration.

In addition, the compositions of this invention may further include one or more accessory ingredients selected from diluents, buffers, flavoring agents, binders, preservatives, and the like.

The features and advantages of the invention are more fully shown by the following illustrative and non-limiting examples.

### EXAMPLE 1

A seven-and-a-half year-old male patient with autism for at least four years had substantial difficulties learning to speak, read, write and process language. The patient orally administered approximately 4 g of the anti-fungal composition of the present invention per day with water. Within three weeks, the patient learned to ride a two-wheel bicycle for the first time. After six weeks, the patient's speech and his ability to read, write and process language improved substantially and his self-stimulating behaviors decreased.

### EXAMPLE 2

A six year-old female patient with autism who had ceased verbalizing with her family, orally administered approximately 2 g of the anti-fungal composition of the present invention per day with water. Within a few days, she started speaking again. Following four months of administration, her verbal improvement leveled off, at which point the dosage was increased to approximately 4 g of the anti-fungal composition per day with water.

### EXAMPLE 3

A six year-old female patient with autism orally administered approximately 0.5 g of the anti-fungal composition of the present invention per day with water. Following administration of the composition for four days, the patient experienced some constipation, which was treated. The patient displayed a large improvement in eye contact and verbal skills. Four weeks later, the patient continued to show improvement.

### EXAMPLE 4

A five year-old male patient with autism and a leaky gut orally administered approximately 2 g of the anti-fungal composition of the present invention per day with water. The patient's gut began to heal immediately with a concomitant jump in verbal articulation. In addition, the patient's gluten infractions did not manifest themselves as they typically did.

### EXAMPLE 5

A three year-old male patient with autism orally administered approximately 2 g of the anti-fungal composition of the present invention per day with water. The dosage was increased up to approximately 4 g of the anti-fungal composition per day and is presently maintained at approximately 2 g of the anti-fungal composition per day. Immediately, the patient began making better eye contact with others and responded to verbal requests from his mother. At the age of four, the diagnosis of autism was completely removed and the patient has entered traditional schools.

### EXAMPLE 6

A three-and-a-half year-old female patient with autism and redness and itching associated with the autism orally administered approximately 2 g of the anti-fungal composition of the present invention per day with water. The patient began repeating sounds and words, and responded to verbal requests from her parents. In addition, the redness and itching subsided and the patient was successfully potty trained for the first time without complaint.

### EXAMPLE 7

A six-and-a-half year-old male patient with autism for at least four years orally administered approximately 2 g of the anti-fungal composition of the present invention per day with water about one year ago (at age 5.5 years). Following thirty days of approximately 2 g/day of the anti-fungal composition, the dosage was increased to approximately 4-6 g/day of the anti-fungal composition for the next thirty days. Thereafter, the dosage was reduced back to approximately 2 g/day of the anti-fungal composition for maintenance. The patient is no longer diagnosed as learning disabled and he is able to attend traditional schools.

### EXAMPLE 8

A four year-old male patient with autism was orally administered approximately 2 g of the anti-fungal composition of the present invention per day with water. The dosage was increased up to approximately 4 g of the anti-fungal composition per day. At the age of five, the diagnosis of autism was completely removed and the patient has entered traditional schools.

### EXAMPLE 9

A 48 year-old female patient with fibromyalgia who did massage therapy, yoga and Pilates, but failed to be relieved of the pain, orally administered approximately 2 g of the anti-fungal composition of the present invention per day with water for two months. The patient's energy level increased substantially and she was able to play eighteen holes of golf on four consecutive days, was able to ride a bicycle sixteen miles and take several 5-6 mile hikes all within the same week, without exhaustion or extreme pain.

### EXAMPLE 10

A 26 year-old female patient with fibromyalgia orally administered approximately 2 g of the anti-fungal composition of the present invention per day with water. Within just four days, the patient noticed a substantial improvement, whereby the pain associated with the condition decreased and her night sweats disappeared.

### EXAMPLE 11

A 55 year-old female patient with fibromyalgia and allergies orally administered approximately 6 g of the anti-fungal composition of the present invention per day with water for about five months. She noticed a significant improvement of her fibromyalgia symptoms and a decrease in the severity of her allergies.

### EXAMPLE 12

A 34 year-old female patient who had suffered fibromyalgia for at least five years orally administered approximately 4 g of the anti-fungal composition of the present invention per day with water. Within three weeks of taking the composition, the patient experienced enough relief of her symptoms to stop taking sleeping pills and pain medications.

### EXAMPLE 13

A 49 year-old female patient with fibromyalgia orally administered approximately 6 g of the anti-fungal composition of the present invention per day with water. Her fibromyalgia is now in a reversed state, whereby her tender points are non-existent and she was able to exercise twenty five to forty five minutes per day.

### EXAMPLE 14

An approximately twenty-five year-old female patient with a severe sinus infection and acid reflux orally administered approximately 2 g of the anti-fungal composition of the present invention per day with water. Both the sinus infection and the acid reflux were completely treated.

### EXAMPLE 15

A 47 year-old female patient with acid reflux orally administered approximately 2 g of the anti-fungal composition of the present invention per day with water for five days and than increased the dosage to approximately 4 g of the anti-fungal composition per day for the next ten days. The heartburn caused by the acid reflux improved and other indications, such as hot flashes and sores on her head, ceased.

### EXAMPLE 16

A 58 year-old female patient with hearing problems and some tinnitus orally administered approximately 2 g of the anti-fungal composition of the present invention per day with water. After experiencing some bowel pains, she increased the dosage of the anti-fungal composition to approximately 4 g per day and started feeling much better. She regained almost all of the hearing in her right ear and the ringing in her ears subsided.

### EXAMPLE 17

A 51 year-old female patient with hearing problems, irritable bowel syndrome, and high blood sugar orally administered approximately 2 g of the anti-fungal composition of the present invention per day with water. She noticed an improvement in her irritable bowel symptoms and her blood sugar problems became more regulated, whereby she didn't become faint and panicky if she was unable to eat at the same time everyday. In addition, although she still needed to wear two digital hearing aids, she noticed an improvement in her hearing whereby she began to hear noises, e.g., alarm clocks that were previously unheard.

### EXAMPLE 18

A 36 year-old female patient with irritable bowel syndrome, tinnitus and depression orally administered approximately 2 g of the anti-fungal composition of the present invention per day with water for three months. She noticed a significant improvement in her irritable bowel symptoms and went off her anti-depression medications. In addition, the noise in her ears ceased.

### EXAMPLE 19

A 50 year-old female patient with adult onset asthma and allergies orally administered approximately 2 g of the anti-fungal composition of the present invention per day with water for several weeks. Her asthma went away completely and she experienced considerable relief from her allergies.

### EXAMPLE 20

A 37 year-old female patient with candidiasis and seasonal hayfever orally administered approximately 2 g of the anti-fungal composition of the present invention per day with water for six months. For the first time in twenty years, the patient did not suffer from seasonal hayfever.

### EXAMPLE 21

A 38 year-old female patient with chronic ear infections and hearing fluctuations over more than twenty years orally administered approximately 2 g of the anti-fungal composition of the present invention per day with water. She experienced relief from her recurrent ear infections and her hearing improved.

### EXAMPLE 22

A 33 year-old female patient with a persistent vaginal yeast infection vaginally administered a gelatin capsule containing approximately 2 g of the anti-fungal composition of the present invention. After several administrations, the yeast infection went away.

### EXAMPLE 23

A 24 year-old female patient with a persistent vaginal itch vaginally administered two gelatin capsules containing approximately 4 g of the anti-fungal composition of the present invention. Immediately, the itching subsided.

### EXAMPLE 24

A 52 year-old female patient with Type I diabetes orally administered approximately 6 g of the anti-fungal composition of the present invention per day with water. She noticed that she was feeling more energetic and that her blood sugars had stabilized.

### EXAMPLE 25

A 55 year-old female patient with Multiple Sclerosis orally administered approximately 4 g of the anti-fungal composition of the present invention per day with water for sixteen days. Prior to taking the anti-fungal composition, the patient could not walk properly and she experienced tingling and numbness on her right side and pressure in her head. After just one day, the pressure in her head eased and the leg muscles spasms became less painful. She began to use her right hand to carry objects without dropping them. She could also walk around the house without help and did not tire as easily.

### EXAMPLE 26

A questionnaire was provided to 500 individual users of antifungal bacterial compositions of the invention. The questionnaire inquired about the disease state or physiological condition afflicting the individual, their gender, how much of the anti-fungal composition they take, how long they have been taking the composition, and their self-determined percent

TABLE 1

| Disease State/ | | | |
|---|---|---|---|
| Adverse | | Dosage | |
| Physiological | # of Respondents | Adminis- | Percent |
| Condition | (Male or Female) | tered/day | Improvement |
| autism | 4 M | ≈2 g to ≈6 g | 1 (less than 50%) |
| | | | 3 (90%-100%) |
| delayed | 1 F; 1 M | ≈4 g | 1 (less than 50%) |
| development | | | 1 (90%-100%) |
| acid reflux | 15 F; 1 M | ≈2 g to ≈8 g | 2 (less than 50%) |
| | | | 1 (50%-60%) |
| | | | 8 (70%-80%) |
| | | | 5 (90%-100%) |
| impaired | 6 F; 3 M | ≈2 g to ≈6 g | 1 (less than 50%) |
| hearing | | | 3 (70%-80%) |
| | | | 5 (90%-100%) |
| chronic ear | 17 F; 1 M | ≈2 g to ≈8 g | 1 (less than 50%) |
| infections | | | 4 (50%-60%) |
| | | | 5 (70%-80%) |
| | | | 8 (90%-100%) |
| seasonal | 30 F; 5 M | ≈2 g to ≈10 g | 4 (less than 50%) |
| allergies | | | 6 (50%-60%) |
| | | | 10 (70%-80%) |
| | | | 15 (90%-100%) |
| fibromyalgia | 33 F; 3 M | ≈2 g to ≈10 g | 12 (less than 50%) |
| | | | 5 (50%-60%) |
| | | | 10 (70%-80%) |
| | | | 9 (90%-100%) |
| Crohn's | 2 F | ≈2 g to ≈4 g | 2 (50%-60%) |
| Disease | | | |
| colitis | 3 F; 1 M | ≈2 g to ≈8 g | 2 (less than 50%) |
| | | | 1 (50%-60%) |
| | | | 1 (70%-80%) |
| IBS | 28 F; 3 M | ≈2 g to ≈10 g | 10 (less than 50%) |
| | | | 5 (50%-60%) |
| | | | 10 (70%-80%) |
| | | | 6 (90%-100%) |
| interstitial | 2 F; 1 M | ≈2 g to ≈10 g | 3 (70%-80%) |
| cystitis | | | |
| acne | 3 F | ≈4 g to ≈8 g | 1 (50%-60%) |
| | | | 2 (70%-80%) |
| sinusitis | 4 F; 2 M | ≈2 g to ≈10 g | 1 (less than 50%) |
| | | | 1 (50%-60%) |
| | | | 2 (70%-80%) |
| | | | 1 (90%-100%) |
| rheumatoid | 3 F; 1 M | ≈2 g to ≈4 g | 4 (90%-100%) |
| arthritis | | | |
| chronic fatigue | 13 F; 2 M | ≈2 g to ≈10 g | 1 (less than 50%) |
| | | | 2 (70%-80%) |
| | | | 12 (90%-100%) |
| asthma | 3 F; 1 M | ≈2 g to ≈8 g | 4 (90%-100%) |
| ADD | 4 F; 4 M | ≈4 g to ≈8 g | 1 (50%-60%) |
| | | | 2 (70%-80%) |
| | | | 1 (90%-100%) |
| rosacea | 5 F | ≈2 g to ≈8 g | 1 (less than 50%) |
| | | | 1 (50%-60%) |
| | | | 2 (70%-80%) |
| | | | 1 (90%-100%) |

It is noted that the administration schedule and dosage varied from patient to patient depending on the severity of the disease state or physiological condition and as such, it was not possible to correlate the percent improvement to the amount of composition taken or the length of time the composition was taken. As discussed previously, the effective amelioration or regulation of the disease state or physiological condition is completely unrelated to the age or gender of the patient (with the exception of patients less than six years of age).

Referring to Table 1, it can be seen that the anti-fungal composition of the present invention effectively treated various disease states and physiological conditions, including, but not limited to, autism, delayed development, acid reflux disease, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, asthma, attention deficit disorder, and rosacea.

While the invention has been described herein with reference to various specific embodiments, it will be appreciated that the invention is not thus limited, and extends to and encompasses various other modifications and embodiments, as will be appreciated by those ordinarily skilled in the art. Accordingly, the invention is intended to be broadly construed and interpreted, in accordance with the ensuing claims.

What is claimed is:

1. A method of treatment or prophylaxis of a disease state or a physiological condition selected from the group consisting of autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, attention deficit disorder, attention deficit/hyperactivity disorder, rosacea, multiple sclerosis, hyperglycemia, and Ménière's disease, said method comprising administering to a subject in need thereof, an effective amount of a composition comprising at least one of (1) Bacillus subtilis, (2) Lactobacillus sporogenes, and (3) Streptococcus faecalis, with the proviso that when the disease state is autism, colitis or Crohn's Disease, the composition comprises Bacillus subtilis, Lactobacillus sporogenes, and Streptococcus faecalis.

2. The method of claim 1, wherein the composition comprises Bacillus subtilis, Lactobacillus sporogenes, and Streptococcus faecalis.

3. The method of claim 1, wherein the method of administration is selected from the group consisting of oral, subcutaneous, intramuscular, intradermal, transdermal, intraocular, intraperitoneal, mucosal, vaginal, rectal, and intravenous.

4. The method of claim 1, wherein an effective dose of said composition is administered at least once a day.

5. The method of claim 1, wherein an effective dose of said composition is administered at least once a week.

6. The method of claim 1, wherein an effective dose of said composition is from about 1.0 g to about 15.0 g.

7. The method of claim 1, wherein an effective dose of said composition is from about 2.0 g to about 10.0 g.

8. The method of claim 1, wherein said composition is a solid, liquid or gas.

9. The method of claim 1, wherein an effective dose is encapsulated for administration.

10. The method of claim 1, wherein an effective dose is administered in a powder form.

11. A method of treatment or prophylaxis of a disease state or a physiological condition selected from the group consisting of delayed development, acid reflux disease, impaired hearing, chronic ear infections, seasonal allergies, Fibromyalgia syndrome, irritable bowel syndrome, attention deficit disorder, attention deficit/hyperactivity disorder, rosacea, multiple sclerosis, hyperglycemia, and Ménière's disease, said method comprising administering to a subject in need thereof, an effective amount of a composition comprising at least one of (1) Bacillus subtilis, (2) Lactobacillus sporogenes, and (3) Streptococcus faecalis.

12. The method of claim 11, wherein the composition comprises Bacillus subtilis, Lactobacillus sporogenes, and Streptococcus faecalis.

13. The method of claim 11, wherein the method of administration is selected from the group consisting of oral, subcutaneous, intramuscular, intradermal, transdermal, intraocular, intraperitoneal, mucosal, vaginal, rectal, and intravenous.

14. A method of treatment or prophylaxis of a disease state or a physiological condition selected from the group consisting of autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, attention deficit disorder, attention deficit/hyperactivity disorder, rosacea, multiple sclerosis, hyperglycemia, and Ménière's disease, said method comprising administering to a subject in need thereof, an effective amount of a composition comprising at least one of (1) Bacillus subtilis, (2) Lactobacillus sporogenes, and (3) Streptococcus faecalis, wherein the method of administration is selected from the group consisting of oral, subcutaneous, intramuscular, intradermal, transdermal, intraocular, intraperitoneal, mucosal, vaginal, rectal, and intravenous, and when the disease state is autism, colitis or Crohn's Disease, the composition comprises Bacillus subtilis, Lactobacillus sporogenes, and Streptococcus faecalis.

15. A method of treatment or prophylaxis of a disease state or a physiological condition selected from the group consisting of autism, delayed development, acid reflux disease, vaginal yeast infections, impaired hearing, chronic ear infections, seasonal allergies, Fibromyalgia syndrome, Crohn's disease, colitis, irritable bowel syndrome, interstitial cystitis, acne, sinusitis, rheumatoid arthritis, chronic fatigue syndrome, asthma, attention deficit disorder, attention deficit/hyperactivity disorder, rosacea, multiple sclerosis, hyperglycemia, and Ménière's disease, said method comprising administering to a subject in need thereof, an effective amount of a composition comprising at least one of (1) Bacillus subtilis, (2) Lactobacillus sporogenes, and (3) Streptococcus faecalis, with the proviso that when the disease state or physiological condition comprises asthma, the composition is administered by an administration modality not including mucoadministration to the airways of the subject.

16. The method of claim 15, wherein said administering step comprises oral administration.

17. The method of claim 15, wherein the administering step comprises a mode of administration selected from the group consisting of subcutaneous, intramuscular, intradermal, transdermal, intraocular, intraperitoneal, mucosal, vaginal, rectal, and intravenous.

18. The method of claim 15, wherein said disease state or physiological condition comprises autism, and said composition consists essentially of (1) Bacillus subtilis, (2) Lactobacillus sporogenes, and (3) Streptococcus faecalis.

19. The method of claim 15, wherein said disease state or physiological condition comprises a disorder selected from the group consisting of autism, attention deficit disorder, and attention deficit/hyperactivity disorder, and said composition comprises (1) Bacillus subtilis, (2) Lactobacillus sporogenes, and (3) Streptococcus faecalis.

20. The method of claim 15, wherein said disease state or physiological condition comprises a disorder selected from the group consisting of colitis and Crohn's disease.

21. The method of claim 20, wherein said composition comprises (1) Bacillus subtilis, (2) Lactobacillus sporogenes, and (3) Streptococcus faecalis.

22. The method of claim 20, wherein the administering step comprises a non-mucosal mode of administration.

23. The method of claim 15, wherein said disease state or physiological condition comprises fibromyalgia.

24. The method of claim 23, wherein said composition comprises at least one of Lactobacillus sporogenes, and Streptococcus faecalis.

25. The method of claim 15, wherein said disease state or physiological condition comprises asthma.

26. The method of claim 15, wherein said composition comprises two or more of (1) Bacillus subtilis, (2) Lactobacillus sporogenes, and (3) Streptococcus faecalis.

27. The method of claim 15, wherein said composition comprises (1) Bacillus subtilis, (2) Lactobacillus sporogenes, and (3) Streptococcus faecalis.

28. The method of claim 15, wherein the composition is administered to the subject in a dose form comprising from about 1.0 to about 15.0 grams of the composition.

29. The method of claim 15, wherein the composition is administered to the subject at a dosage of from about 2.0 to about 10.0 grams.

30. The method of claim 15, wherein the composition is administered to the subject at least once weekly.

31. The method of claim 15, wherein the composition is administered to the subject at least once daily.

32. The method of claim 15, wherein the composition is administered to the subject in a capsulized dose form.

33. The method of claim 15, wherein the composition is administered to the subject in a powder form.

34. A method of treatment or prophylaxis of autism, said method comprising administering to a subject in need thereof, an effective amount of a composition comprising (1) Bacillus subtilis, (2) Lactobacillus sporogenes, and (3) Streptococcus faecalis.
